(19) United States
(12) Patent Application Publication
(10) Pub. No.: US 2016/0360988 A1
(43) Pub. Date: Dec. 15, 2016

(54) ECG LEADS

(71) Applicant: IGOR ABRAMOV, Vista, CA (US)

(72) Inventor: IGOR ABRAMOV, Vista, CA (US)

(21) Appl. No.: 15/135,550

(22) Filed: Apr. 22, 2016

Related U.S. Application Data
(60) Provisional application No. 62/175,836, filed on Jun. 15, 2015.

(51) Int. Cl. A61B 5/0408; A61B 5/00

(52) U.S. Cl. CPC A61B 5/0408; A61B 5/684; A61B 2562/08

(57) ABSTRACT

The present invention provides a positive identification of each ECG lead (12,16), both visually under varying illumination conditions as well as by touch only, including identification with gloved hands.

In addition, in several embodiments (20, 24) the lead anthropomorphic shape unambiguously indicates the lead placement on patient's body.

This construction and features of the present invention enable fast and accurate identification of the ECG leads and ensure their correct placement on the patient's body.

### FIELD OF INVENTION

This invention relates in general to electrocardiogram (ECG) leads and particularly to improved designs of such leads.

### BACKGROUND OF INVENTION

Electrocardiogram (ECG) leads are used to connect ECG electrodes placed on the patient to the ECG monitoring system.

Most conventional ECG leads fall into two categories. The ones in the first category contain a snap-on spring contact which engages a protruding conductor pin on an ECG electrode pad. The spring contact is electrically connected to a cable leading to the ECG monitoring system. The spring contact itself and its cable interface are encapsulated in plastic to provide mechanical integrity to the junction between the contact and the cable, create a strain relief for the cable, and create a grasping surface to facilitate user's attaching and detaching the lead from the electrode pad.

The leads in another category include various types of clamping connectors which engage conductors on the ECG electrode pads.

The number of physical ECG leads connected to the patient varies between 3 and 10 depending on the sophistication of the ECG monitoring system.

The placement of the lead-electrode pad pairs on a patient is important in obtaining accurate ECG waveform readout and a subsequent correct medical evaluation of patient's condition.

An improper lead-electrode positioning can lead to misinterpretation of the ECG signals and potentially wrong medical diagnosis of patient's condition.

For example, an accidental mis-positioning of the limb lead electrodes is a common cause of ECG trace abnormality and may simulate pathology such as ectopic atrial rhythm, chamber enlargement or myocardial ischemia and infarction.

To help with ECG leads' identification and their proper placement, the leads are customarily color-coded and additionally may contain alphanumeric labels or inscriptions.

However, these features are often inadequate in ensuring proper identification of the leads and their subsequent proper placement.

For example, printed labels are attached to the top of the leads by adhesives and can be obliterated, fade, made otherwise unreadable, or get detached altogether. Molded-in inscriptions are sometimes located on the top of leads and by necessity are small since they have to fit on a small lead head. They are generally shallow, can be damaged or obliterated altogether, and can be hard to read, since they are of the same color as the rest of the lead.

Inscriptions which are directly printed onto the lead with inks or paints, in addition to being by necessity small, can also be obliterated, fade, or change/lose their contrast and readability under some illuminating conditions.

With color-coded leads without inscriptions, or where the inscriptions are unreadable, medical personnel have to rely on the lead color alone for the lead placement.

Since the color-coding standards of ECG leads vary between countries, a possibility exists to mis-position leads if the equipment origin is unknown to the administering personnel, as can happen during international relief operations, disaster response or wartime situations.

Additionally, in high stress environments, such as battlefields, mass casualty scenes, disasters and such, medical personnel may forget or confuse the lead color designation which could lead to the lead mis-positioning.

Since mis-positioned leads would occur in pairs, medical personnel has to be in close proximity to and have enough illumination to identify the leads on the patient's body. If there are several pairs of mis-positioned leads in a 10-lead ECG bundle, the task can be quite challenging and time-consuming, especially under field conditions.

Color-coded leads also present identification problem in cases of:
a) color-blind administering personnel;
b) completely or partially blind or blinded administering personnel;
c) low illumination conditions, including total darkness;
d) mono-chromatic-, or near-monochromatic illumination, such as produced by some outdoors lighting sources, such as sodium vapor lamps, or found in the interiors of vehicles, vessels or aircraft which may use internal safety or battlefield red light illumination. For example, red and white colors may look the same under red illumination, and so can black and green colors.

In addition, in some emergency situations untrained medical- or civilian personnel may be called upon to attach the ECG electrode pads and their respective leads to a patient so that a patient's ECG can be acquired and transmitted for remote evaluation. In these situations a probability of the electrode-lead mis-positioning could rise multifold with the present lead designs.

At present, neither the leads with snap-on connectors, nor more sophisticated leads with specialized connector clips offer differentiation in shape among the leads to be placed in various locations on a patient's body.

Therefore, there exists a need for improved ECG leads which would be easier to identify under various lighting conditions, including total darkness, and by an un-trained medical or civilian personnel, or medical personnel operating in high-stress situations.

For lead shapes with relatively large features, identification by touch alone is possible even by personnel wearing thicker protective tactical gloves, not just conventional thin elastomeric medical ones.

Additionally, a shape-enabled and differentiated lead would offer muscle-memory advantage to trained medical personnel operating in high-stress environments, thus improving the accuracy of lead placement and the time required to accomplish it.

### OBJECTIVES OF THE INVENTION

Thus, it is the objective of instant invention to provide an ECG lead which would be identifiable under varying lighting conditions including total darkness.

Another objective is to provide an ECG lead which would be identifiable by touch alone.

Yet another objective is to provide an ECG lead which would be identifiable by touch by a gloved hand.

Another objective is to provide an ECG lead whose identification would not rely on printed inscriptions.

Yet another objective is to provide an ECG lead whose identification would not rely on color.

Another objective is to provide an ECG lead whose target placement on patient's body would be identifiable by its shape.

### SUMMARY OF THE INVENTION

In accordance with the present invention, ECG leads with varying shapes are provided. A particular shape of each lead uniquely identifies the lead and corresponds to its placement on a patient's body. In several embodiments the anthropomorphic shape of the lead itself directly identifies its placement on the patient's body.

### PRIOR ART

The prior art is comprised of conventional cylindrical snap-on or clamp-type leads differentiated by color and sometimes additional printed inscriptions on their upper faces, or color clip-on rings attached onto the lead cables. The shapes of the leads do not differ from each other for either type of the leads.

For example, U.S. Pat. No. 7,844,316 to Botero shows various insignia on the top of the leads, but the leads themselves are of uniform shape.

U.S. Pat. No. 8,408,948 to Selvitelli et al. shows insignia on the clamp-style leads, but again, no differentiation in the shape of the leads is described.

In cases where lead inscriptions are absent, obliterated or degraded, and inability of the administering personnel to either distinguish the colors or match the colors to the lead placement, lead placement errors may result.

The shape-encoding or differentiation of the ECG leads is not described in prior art to-date.

### OBJECTS AND ADVANTAGES

In contrast to the prior art mentioned hereinabove, the present invention provides a positive identification of each ECG lead, both visually under varying illumination conditions as well as by touch only, including identification with gloved hands.

In addition, in several embodiments the lead shape unambiguously indicates the lead placement on patient's body.

This construction and features of the present invention enable fast and accurate identification of the ECG leads and their correct placement on the patient's body.

In addition, the lead features of the present invention enable quick validation of the leads already positioned on the patient's body, including doing so in complete darkness.

Additionally, anthropomorphic-style leads could lead to faster learning of the proper lead placement by the medical personnel.

### BRIEF DESCRIPTION OF THE DRAWINGS

FIG. 1 is a partial perspective view of prior art ECG lead.

FIG. 2 is a partial perspective view of a rectangular prism-shaped ECG lead.

FIG. 3 is a partial perspective view of a hexagonal prism-shaped ECG lead.

FIG. 4 is a partial perspective view of a triangular prism-shaped ECG lead.

FIGS. 5a, 5b are partial perspective views of an ECG lead shaped like characters denoting lead placement.

FIGS. 6a, 6b are partial perspective views of an ECG lead shaped like a partial human torso with lead placement indicators.

FIGS. 7a, 7b are partial perspective views of an ECG lead shaped like a partial human body with lead placement indicators

### DESCRIPTION OF THE EMBODIMENTS

In the foregoing description like components are referenced by the like numerals.

Prior art ECG lead is shown in FIG. 1. Cylindrical contact head 2 is connected to cable 6 and to electrode 4.

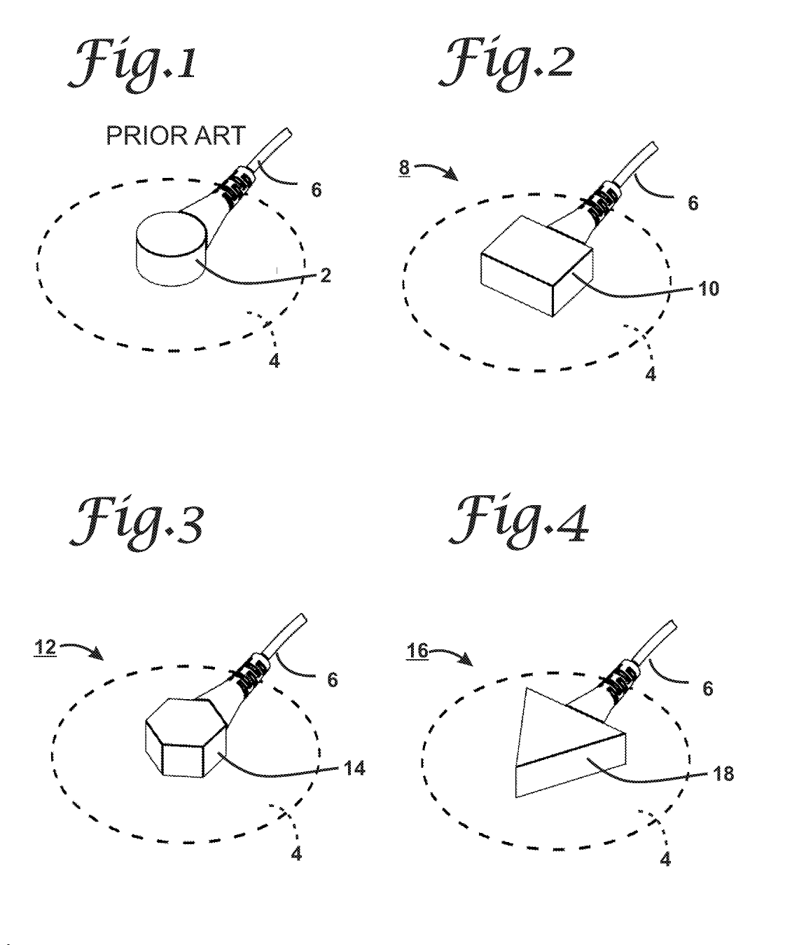

ECG lead embodiment 8 on FIG. 2 comprises a generally rectangular prismatic body 10 connected to cable 6 and to electrode 4. An internal snap-on contact is not shown.

Embodiment 12 on FIG. 3 comprises a generally hexagonal prismatic body 14 connected to cable 6 and to electrode 4. An internal snap-on contact is not shown.

FIG. 4 shows ECG lead embodiment 16 which comprises a generally triangular prismatic body 18 connected to cable 6 and to electrode 4. An internal snap-on contact is not shown.

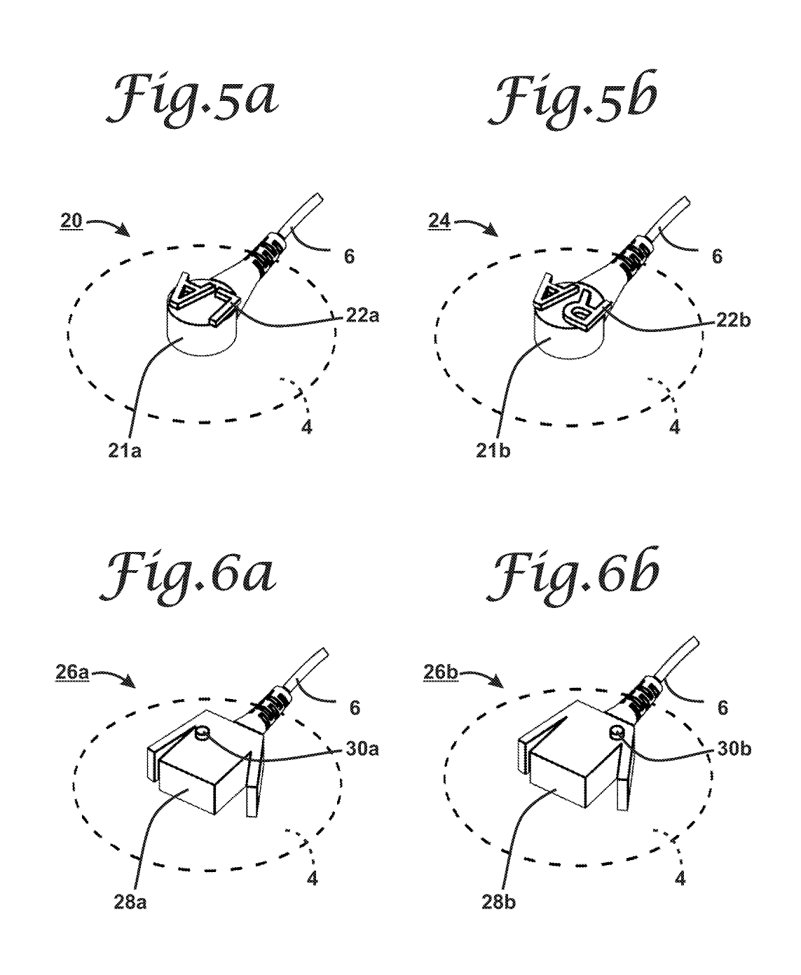

FIG. 5a shows ECG lead embodiment 20 which comprises base 21a which in turn comprises integral position indicator 22a specifying the position of the lead, in this case ‘LEFT ARM’ (‘LA’). An internal snap-on contact is not shown.

FIG. 5b shows ECG lead embodiment 24 which comprises base 21b which in turn comprises integral position indicator 22b specifying the position of the lead, in this case ‘RIGHT ARM’ (‘RA’). An internal snap-on contact is not shown.

FIG. 6a shows ECG lead embodiment 26a which comprises body 28a which is a stylized partial human torso, which further comprises integral raised position indicator 30a specifying position of the lead on the patient's torso. An internal snap-on contact is not shown.

FIG. 6b shows ECG lead embodiment 26b which comprises body 28b which is a stylized partial human torso, which further comprises raised integral position indicator 30b specifying position of the lead on the patient's torso. An internal snap-on contact is not shown.

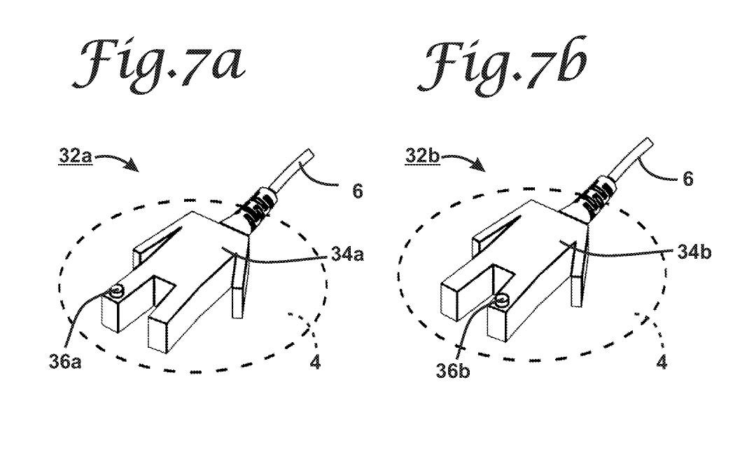

FIG. 7a shows ECG lead embodiment 32a which comprises body 34a which is a stylized partial human body, which further comprises integral raised position indicator 36a specifying position of the lead on the patient's body. An internal snap-on contact is not shown.

FIG. 7b shows ECG lead embodiment 32b which comprises body 36b which is a stylized partial human body, which further comprises integral raised position indicator 36b specifying position of the lead on the patient's body. An internal snap-on contact is not shown.

### OPERATION

For embodiments 8, 12, 16, 20, and 24, prior to the lead placement on the patient's body, administering personnel can easily distinguish the overall shape of the lead visually and even by touch alone if required, and ensure its proper placement on patient's body. For embodiments 26a, 26b, 32a and 32b prior to the lead placement on the patient's body, administering personnel can easily recognize the intended position of the lead visually and even by touch alone by sensing integral raised position indicators 30a, 30b, 36a, 36b, respectively.

### ADDITIONAL EMBODIMENTS

Although descriptions provided above contain many specific details, they should not be construed as limiting the scope of the present invention.

In addition to the shape differentiation, the leads may (and indeed, should) retain their color coding, since this color coding is standardized by several regulatory bodies.

Conventional labels and imprinting can also be added for enhanced utility.

Particular shapes of the leads can be quite diverse. For example, they can be star-like with several extending rays; they can be gear-like, and similar.

The leads may have different surface textures and patterns: for example rough for the ‘RA’ lead and smooth for the ‘LA’ one.

Also, stylized human body shapes can have varying appearance, selected for ergonomics and operational modalities. For example, rounded corners could be utilized to minimize potential snagging of the lead on clothing, bed linens, or various lines or dressings.

Several indicia can be included on the common shape, like a torso, with one indicator more prominent than the others, to denote its relative position within the group of indicia. This is valuable, for example, in case of a 12-lead ECG where electrodes V1 through V6 are positioned close to each other.

Separate stylized human body parts can be utilized as well, for example a right arm and shoulder for the ‘RA’ lead.

Thus, the scope of this invention should be determined from the appended claims and their legal equivalents.

I claim:

1. An electrode lead for electrical connection to a body, said lead terminating in an electrical contact, said lead comprising an identifying portion, said identifying portion further comprising a guide shape corresponding to said lead's placement location on said body.

2. The electrode lead of claim 1 wherein said guide shape is graspable by a user.

3. The lead of claim 1, wherein said shape comprises at least one essentially geometric shape.

4. The lead of claim 1, wherein said shape comprises at least one alphanumeric character.

5. The lead of claim 1, wherein said shape comprises at least one logogram character.

6. The lead of claim 1, wherein said shape comprises at least one syllabic character.

7. The lead of claim 1, wherein said shape comprises an essentially anthropomorphic shape.

8. The lead of claim 7 wherein said shape further comprises at least one lead placement indicator corresponding to the said lead placement on said body.

9. The placement indicator of claim 8 comprising at least one tactile feature.

10. An electrode lead for electrical connection to a body, said lead terminating in an electrical contact, said lead comprising an identifying portion, said identifying portion further comprising a guide shape corresponding to said lead's placement location on said body, said shape graspable by a user.

11. A method of placing an electrical lead onto its intended location on a body by:
a) encoding the intended position of said lead in an essentially 3-dimensional shape of said lead, said shape graspable by user;
b) user identifying said shape at the point of use;
c) user grasping said shape;
d) user connecting said lead to an electrocardiogram electrode patch and placing said patch onto said body at a location indicated by said shape.

12. The lead of claim 11, wherein said shape comprises at least one geometric shape.

13. The lead of claim 11, wherein said shape comprises at least one alphanumeric character.

14. The lead of claim 11, wherein said shape comprises at least one logogram character.

15. The lead of claim 11, wherein said shape comprises at least one syllabic character.

16. The lead of claim 11, wherein said shape comprises an essentially anthropomorphic form.

17. The lead of claim 16 wherein said shape further comprises at least one lead placement indicator corresponding to the said lead placement on said body.

18. The placement indicator of claim 17 comprising at least one tactile feature
